SEQUENCE LISTING (3 sequences)

SEQ ID NO: 1 - DNA, 23 bp, Artificial Sequence
  Synthetic Polypeptide
caaggtcatccatgacaactttg

SEQ ID NO: 2 - DNA, 19 bp, Artificial Sequence
  Synthetic Polypeptide
gggccatccacagtcttct

SEQ ID NO: 3 - DNA, 25 bp, Artificial Sequence
  Synthetic Polypeptide
  misc_feature (1)..(1): Hexachlorofluorescein
  misc_feature (3)..(3): Black Hole Quencher-1
accacagtccatgccatcactgcca

(19) United States
(12) Patent Application Publication
(10) Pub. No.: US 2021/0189397 A1
(43) Pub. Date: Jun. 24, 2021

(54) SELF-MANAGEABLE ABNORMAL SCAR TREATMENT WITH SPHERICAL NUCLEIC ACID (SNA) TECHNOLOGY

(71) Applicant: NORTHWESTERN UNIVERSITY, Evanston, IL (US)

(72) Inventors: Chad A. Mirkin, Wilmette, IL (US); Anthony J. Sprangers, Evanston, IL (US); Shengshuang Zhu, Evanston, IL (US); Adam J. Ponedal, Evanston, IL (US); Timothy J. Merkel, San Diego, CA (US); Suguna P. Narayan, Evanston, IL (US)

(21) Appl. No.: 17/054,441

(22) PCT Filed: May 10, 2019
(86) PCT No.: PCT/US19/31797

§ 371 Date: Nov. 10, 2020

Related U.S. Application Data
(60) Provisional application No. 62/669,768, filed on May 10, 2018.

(51) Int. Cl. C12N 15/113; A61P 17/02; A61K 9/00; A61K 9/127; A61K 47/69; A61K 47/54; A61K 47/68

(52) U.S. Cl. CPC C12N 15/1136; A61P 17/02; A61K 9/0014; A61K 9/1271; A61K 47/6937; A61K 47/6923; C12N 2310/14; A61K 47/6911; A61K 47/6849; C12N 2310/3519; C12N 2320/32; C12N 2320/31; C12N 2310/11; A61K 47/549

(57) ABSTRACT

The disclosure is related to compositions and methods comprising spherical nucleic acids (SNAs) and their use in penetrating skin and inhibiting gene expression to develop a scar treatment.

### CROSS-REFERENCE TO RELATED APPLICATION

This application claims the priority benefit under 35 U.S.C. § 119(e) of U.S. Provisional Patent Application No. 62/669,768, filed May 10, 2018, the disclosure of which is incorporated herein by reference in its entirety.

### INCORPORATION BY REFERENCE OF MATERIAL SUBMITTED ELECTRONICALLY

This application contains, as a separate part of the disclosure, a sequence listing in computer-readable form which is incorporated by reference in its entirety and identified as follows: Filename: 2018-071_Seqlisting.txt; Size: 1,032 bytes, Created: May 10, 2019.

### BACKGROUND

Every year more than 200 million surgeries are performed world-wide, each of which leaves a scar that can potentially develop into permanent abnormal scarring, such as a hypertrophic and keloid scar. Scarring also occurs as a result of burning and accidents. Extreme abnormal scarring can be aesthetically disturbing and mentally stressful.

### SUMMARY

The present disclosure provides compositions and methods in which spherical nucleic acids (SNAs) are exploited to penetrate skin and effectuate potent gene regulation to develop a self-administrable scar treatment.

Accordingly, in some aspects the disclosure provides a method of treating and/or attenuating an abnormal scar in a subject, comprising topically administering a composition to the abnormal scar, the composition comprising: a spherical nucleic acid (SNA) comprising a nanoparticle and an oligonucleotide on the surface of the nanoparticle, wherein topical administration of the SNA inhibits expression of transforming growth factor beta 1 (TGF-β1), thereby treating and/or attenuating the abnormal scar. In some embodiments, the nanoparticle is organic. In further embodiments, the nanoparticle is inorganic. In some embodiments, the nanoparticle is a liposome. In further embodiments, the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, and lipid A. In some embodiments, the oligonucleotide comprises a tocopherol, a cholesterol moiety, DOPE-butamide-phenylmaleimido, or lyso-phosphoethanolamine-butamide-pneylmaleimido.

In further embodiments, the nanoparticle is a micelle. In some embodiments, the nanoparticle is polymeric. In further embodiments, the nanoparticle comprises poly (lactic-co-glycolic acid) (PLGA). In some embodiments, the nanoparticle is metallic. In further embodiments, the nanoparticle is a colloidal metal. In still further embodiments, the nanoparticle is selected from the group consisting of a gold nanoparticle, a silver nanoparticle, a platinum nanoparticle, an aluminum nanoparticle, a palladium nanoparticle, a copper nanoparticle, a cobalt nanoparticle, an indium nanoparticle, and a nickel nanoparticle.

In some embodiments, the oligonucleotide is bound to said nanoparticle through one or more sulfur linkages. In further embodiments, the oligonucleotide is from about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, or about 5 to about 10 nucleotides in length. In still further embodiments, the oligonucleotide comprises RNA or DNA. In some embodiments, the RNA is selected from the group consisting of a small inhibitory RNA (siRNA), a single-stranded RNA (ssRNA) that forms a triplex with double stranded DNA, and a ribozyme. In some embodiments, the RNA is a microRNA. In further embodiments, the DNA is antisense-DNA or a catalytically active DNA molecule (DNAzyme).

In some embodiments, the nanoparticle ranges from about 1 nm to about 250 nm in diameter, about 1 nm to about 240 nm in diameter, about 1 nm to about 230 nm in diameter, about 1 nm to about 220 nm in diameter, about 1 nm to about 210 nm in diameter, about 1 nm to about 200 nm in diameter, about 1 nm to about 190 nm in diameter, about 1 nm to about 180 nm in diameter, about 1 nm to about 170 nm in diameter, about 1 nm to about 160 nm in diameter, about 1 nm to about 150 nm in diameter, about 1 nm to about 140 nm in diameter, about 1 nm to about 130 nm in diameter, about 1 nm to about 120 nm in diameter, about 1 nm to about 110 nm in diameter, about 1 nm to about 100 nm in diameter, about 1 nm to about 90 nm in diameter, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, or about 1 nm to about 20 nm in diameter, or about 1 nm to about 10 nm in diameter. In some embodiments, the nanoparticle has a diameter of 50 nanometers or less.

In some embodiments, expression of TGF-β1 is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%. In further embodiments, expression of a target contemplated by the disclosure is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

In some embodiments, the oligonucleotide is bound to the nanoparticle at a surface density of at least 10 pmol/cm², at least 15 pmol/cm², at least 20 pmol/cm², at least 10 pmol/cm², at least 25 pmol/cm², at least 30 pmol/cm², at least 35 pmol/cm², at least 40 pmol/cm², at least 45 pmol/cm², or at least 50 pmol/cm². In some embodiments, the nanoparticle comprises from about 50 to about 500 oligonucleotides. In further embodiments, the particle comprises 150 to 350 oligonucleotides. In still further embodiments, the particle comprises 200 to 300 oligonucleotides.

In some embodiments, the SNA further comprises a therapeutic. In further embodiments, the therapeutic is encapsulated in the nanoparticle. In some embodiments, the therapeutic is conjugated to the surface of the nanoparticle. In still additional embodiments, the therapeutic is a small molecule, an additional oligonucleotide, a protein, or a peptide. In some embodiments, the protein is a steroid or an antibody. In further embodiments, the antibody is directed against TGF-β1, TGF-62, connective tissue growth factor (CTGF), an extracellular matrix protein, matrix metallopeptidase 2 (MMP2), metallopeptidase inhibitor 1 (TIMP1), a Smad protein, transforming growth factor beta receptor 1 and 2 (TGFBRI, TGFBRII), or a Bcl-2 family member. In some embodiments, the additional oligonucleotide is siRNA, a ribozyme, antisense DNA, or a catalytically active DNA molecule (DNAzyme). In some embodiments, the antibody is bevancizumab.

In some aspects, the disclosure provides a spherical nucleic acid (SNA) comprising a nanoparticle and an oligonucleotide on the surface of the nanoparticle, wherein the oligonucleotide is sufficiently complementary to one or more portions of a target polynucleotide to hybridize to the target polynucleotide and inhibit expression of a gene product expressed from the target polynucleotide. In various embodiments, the target polypeptide is TGF-β1, connective tissue growth factor (CTGF), an extracellular matrix protein, matrix metallopeptidase 2 (MMP2), metallopeptidase inhibitor 1 (TIMP1), a Smad protein, transforming growth factor beta receptor 1 and 2 (TGFBRI, TGFBRII), or a Bcl-2 family member. In some embodiments, the extracellular matrix protein is fibronectin, collagen, elastin, vitronectin, bone sialoprotein, or laminin.

### BRIEF DESCRIPTION OF THE FIGURES

FIG. 1 shows characterization of the SNAs.

FIG. 2 depicts scar cell uptake of SNAs, and shows that AuSNAs (gold SNAs) are taken up by rabbit and human fibroblasts.

FIG. 3 shows that TGF-β1-targeting SNAs effectively downregulate TGF-β1 in hypertrophic scar cells.

FIG. 4 shows that TGF-β1-targeting SNAs potently downregulate TGF-β1 and its downstream growth factor in rabbit ear models.

FIG. 5 depicts clinical pictures of abnormal scar after SNA treatment.

FIG. 6 shows a further depiction of the characterization of the SNAs.

FIG. 7 shows results of experiments designed to screen antisense DNA that knocks down TGF-β1.

FIG. 8 shows the uptake profile of AuSNAs into three model cell lines.

FIG. 9 shows TGF-β1 reduction in patient-derived keloid scar cells.

FIG. 10 depicts the experimental protocol for testing SNA efficacy to reduce abnormal scarring in a rabbit ear model.

FIG. 11 shows that potent TGF-β1 knockdown was achieved using both SNA constructs (i.e., both the AuSNA and the liposomal SNA (LSNA)).

FIG. 12 shows that treatment with both SNA constructs (i.e., both the AuSNA and the liposomal SNA (LSNA)) resulted in reduced scar elevation.

FIG. 13 shows that SNA treatment leads to collagen reformation.

FIG. 14 shows graphical depictions of exemplary AuSNAs and LSNAs.

### DETAILED DESCRIPTION

Despite advances in understanding the molecular mechanism pertaining to scar formation and decades of development of scar care, an effective, self-manageable scar treatment is still lacking. Accordingly, the present disclosure provides compositions and methods comprising spherical nucleic acids (SNAs) and their use in penetrating skin and inhibiting gene expression to develop a self-administrable scar treatment.

As used herein, the term “attenuate” means to allow for wound closure that results in a less scarred character. In some embodiments, attenuating a scar applies to the case in which a composition of the disclosure arrests the development of a fresh scar as it continues to grow after wound closure.

As used herein, “treating” and “treatment” refers to any reduction in the severity and/or onset of symptoms associated with an abnormal scar. Accordingly, “treating” and “treatment” includes therapeutic and prophylactic measures. One of ordinary skill in the art will appreciate that any degree of protection from, or amelioration of, an abnormal scar is beneficial to a subject, such as a human patient. The quality of life of a patient is improved by reducing to any degree the severity of symptoms in a subject and/or delaying the appearance of symptoms.

The terms “polynucleotide” and “oligonucleotide” are interchangeable as used herein.

As used in this specification and the appended claims, the singular forms “a,” “an,” and “the” include plural reference unless the context clearly dictates otherwise.

According to the disclosure, individuals in need of compositions provided herein are, in various embodiments, able to apply this technology to treat their scar by themselves without visiting a hospital in a self-manageable, painless manner.

Spherical Nucleic Acids. Spherical nucleic acids (SNAs) comprise densely functionalized and highly oriented polynucleotides on the surface of a nanoparticle which can either be inorganic (such as gold, silver, or platinum), organic (such as liposomal). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain and blood-tumor barriers as well as the epidermis.

Nanoparticles are therefore provided which are functionalized to have a polynucleotide attached thereto. In general, nanoparticles contemplated include any compound or substance with a high loading capacity for a polynucleotide as described herein, including for example and without limitation, a metal, a semiconductor, a liposomal particle, insulator particle compositions, and a dendrimer (organic versus inorganic).

Thus, nanoparticles are contemplated which comprise a variety of inorganic materials including, but not limited to, metals, semi-conductor materials or ceramics as described in U.S. Patent Publication No. 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles.

Liposomal particles, for example as disclosed in International Patent Application No. PCT/US2014/068429 (incorporated by reference herein in its entirety, particularly with respect to the discussion of liposomal particles) are also contemplated by the disclosure. Hollow particles, for example as described in U.S. Patent Publication No. 2012/0282186 (incorporated by reference herein in its entirety) are also contemplated herein.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO2, Sn, SnO2, Si, SiO2, Fe, Fe+4, Ag, Cu, Ni, Al, steel, cobalt-chrome alloys, Cd, titanium alloys, AgI, AgBr, HgI2, PbS, PbSe, ZnTe, CdTe, In2S3, In2Se3, Cd3P2, Cd3As2, InAs, and GaAs. Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); Ushida et al., J. Phys. Chem., 95, 5382 (1992).

In practice, methods of increasing cellular uptake and inhibiting gene expression are provided using any suitable particle having oligonucleotides attached thereto that do not interfere with complex formation, i.e., hybridization to a target polynucleotide. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles particles, aggregate particles, isotropic (such as spherical particles) and anisotropic particles (such as non-spherical rods, tetrahedral, prisms) and core-shell particles such as the ones described in U.S. patent application Ser. No. 10/034,451, filed Dec. 28, 2002, and International Application No. PCT/US01/50825, filed Dec. 28, 2002, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

Also as described in U.S. Patent Publication No. 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in U.S. Patent Publication No. 20030147966, nanoparticles contemplated are produced using HAuCl4 and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

Polynucleotides. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in European Patent Publication EP 1 072 679 and International Patent Publication No. WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof generally comprise a polynucleotide from about 5 nucleotides to about 100 nucleotides in length. More specifically, nanoparticles are functionalized with a polynucleotide that is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the polynucleotide attached to a nanoparticle is DNA. When DNA is attached to the nanoparticle, the DNA is in some embodiments comprised of a sequence that is sufficiently complementary to a target region of a polynucleotide such that hybridization of the DNA polynucleotide attached to a nanoparticle and the target polynucleotide takes place, thereby associating the target polynucleotide to the nanoparticle. The DNA in various aspects is single stranded or double-stranded, as long as the double-stranded molecule also includes a single strand region that hybridizes to a single strand region of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide.

In some embodiments, the disclosure contemplates that a polynucleotide attached to a nanoparticle is RNA. The RNA can be either single-stranded or double-stranded, so long as it is able to hybridize to a target polynucleotide.

In some aspects, multiple polynucleotides are functionalized to a nanoparticle. In various aspects, the multiple polynucleotides each have the same sequence, while in other aspects one or more polynucleotides have a different sequence. In further aspects, multiple polynucleotides are arranged in tandem and are separated by a spacer. Spacers are described in more detail herein below.

Polynucleotide attachment to a nanoparticle. Polynucleotides contemplated for use in the methods include those bound to the nanoparticle through any means (e.g., covalent or non-covalent attachment). Regardless of the means by which the polynucleotide is attached to the nanoparticle, attachment in various aspects is effected through a 5′ linkage, a 3′ linkage, some type of internal linkage, or any combination of these attachments. In some embodiments, the polynucleotide is covalently attached to a nanoparticle. In further embodiments, the polynucleotide is non-covalently attached to a nanoparticle.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating polynucleotides with a liposomal particle are described in PCT/US2014/068429, which is incorporated by reference herein in its entirety.

Spacers. In certain aspects, functionalized nanoparticles are contemplated which include those wherein an oligonucleotide and a domain are attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In aspects of the invention where a domain is attached directly to a nanoparticle, the domain is optionally functionalized to the nanoparticle through a spacer. In another aspect, the domain is on the end of the oligonucleotide that is opposite to the spacer end. In aspects wherein domains in tandem are functionalized to a nanoparticle, spacers are optionally between some or all of the domain units in the tandem structure. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. As a result of the binding of the spacer to the nanoparticles, the polynucleotide is spaced away from the surface of the nanoparticles and is more accessible for hybridization with its target. In various embodiments, the length of the spacer is or is equivalent to at least about 5 nucleotides, 5-10 nucleotides, 10 nucleotides, 10-30 nucleotides, or even greater than 30 nucleotides. The spacer may have any sequence which does not interfere with the ability of the polynucleotides to become bound to the nanoparticles or to the target polynucleotide. In certain aspects, the bases of the polynucleotide spacer are all adenylic acids, all thymidylic acids, all cytidylic acids, all guanylic acids, all uridylic acids, or all some other modified base. Accordingly, in some aspects wherein the spacer consists of all guanylic acids, it is contemplated that the spacer can function as a domain as described herein.

Nanoparticle surface density. A surface density adequate to make the nanoparticles stable and the conditions necessary to obtain it for a desired combination of nanoparticles and polynucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of polynucleotide on the surface of the SNA is measured by the number of oligonucleotides on the surface of a SNA. With respect to the surface density of oligonucleotides on the surface of a SNA of the disclosure, it is contemplated that a SNA as described herein comprises from about 1 to about 500 oligonucleotides on its surface. In further embodiments, a SNA comprises from about 150 to about 350 oligonucleotides on its surface. In further embodiments, a SNA comprises from about 200 to about 300 oligonucleotides on its surface. In various embodiments, a SNA comprises from about 10 to about 100, or from 10 to about 90, or from about 10 to about 80, or from about 10 to about 70, or from about 10 to about 60, or from about 10 to about 50, or from about 10 to about 40, or from about 10 to about 30, or from about 10 to about 20 oligonucleotides on its surface. In further embodiments, a SNA comprises about, at least about, or less than about 5, 10, 20, 30, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 oligonucleotides on its surface.

In various aspects, the present disclosure provides a method of inhibiting expression of a gene product encoded by a target oligonucleotide comprising contacting the target oligonucleotide with a nanoparticle as described herein under conditions sufficient to inhibit expression of the gene product. In some embodiments, expression of the gene product is inhibited in vivo. In some embodiments, expression of the gene product is inhibited in vitro. In various embodiments, expression of the gene product is inhibited by at least about 5% relative to expression of the gene product in the absence of contacting the target oligonucleotide with the nanoparticle, for example, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, and/or at least about 95%.

In various aspects, the methods include use of an oligonucleotide which is 100% complementary to the target oligonucleotide, i.e., a perfect match, while in other aspects, the oligonucleotide is at least (meaning greater than or equal to) about 95% complementary to the target oligonucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the target oligonucleotide over the length of the oligonucleotide to the extent that the oligonucleotide is able to achieve the desired of inhibition of a target gene product. It will be understood by those of skill in the art that the degree of hybridization is less significant than a resulting detection of the target oligonucleotide, or a degree of inhibition of gene product expression.

Oligonucleotide complementarity. “Hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. Under appropriate stringency conditions, hybridization between the two complementary strands could reach about 60% or above, about 70% or above, about 80% or above, about 90% or above, about 95% or above, about 96% or above, about 97% or above, about 98% or above, or about 99% or above in the reactions. It will be understood by those of skill in the art that the degree of hybridization is less significant than a resulting degree of inhibition of gene product expression.

In various aspects, the methods of the disclosure include use of an oligonucleotide that is 100% complementary to the target polynucleotide, i.e., a perfect match, while in other aspects, the oligonucleotide is at least (meaning greater than or equal to) about 95% complementary to the polynucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to the polynucleotide over the length of the oligonucleotide to the extent that the oligonucleotide is able to achieve the desired degree of inhibition of a target gene product.

In any of the aspects or embodiments of the disclosure, the oligonucleotide utilized in the methods of the disclosure is RNA or DNA. The RNA can be an inhibitory RNA (RNAi) that performs a regulatory function, and in various embodiments is selected from the group consisting of a small inhibitory RNA (siRNA), an RNA that forms a triplex with double stranded DNA, and a ribozyme. Alternatively, the RNA is microRNA that performs a regulatory function. The DNA is, in some embodiments, an antisense-DNA or a DNAzyme. DNAzymes are described, e.g., in Zhou et al., Theranostics 7(4): 1010-1025 (2017), incorporated herein by reference in its entirety.

Therapeutics. A SNA of the disclosure is contemplated, in various aspects and embodiments, to further comprise a therapeutic. The therapeutic may be encapsulated in the SNA, conjugated to the surface of the SNA, administered concurrently with the SNA, or a combination thereof. Any therapeutic that provides an anti-scarring effect is contemplated by the disclosure.

In various embodiments, the therapeutic is a small molecule, an additional oligonucleotide, a protein, or a peptide. In some embodiments, the small molecule is ginsenoside-Rg3. In further embodiments, the protein is a steroid or an antibody. In still further embodiments, the antibody is directed against transforming growth factor beta receptor 1 (TGFBR1). In some embodiments, the additional oligonucleotide is siRNA, a ribozyme, antisense DNA, or DNAzyme. In further embodiments, the oligonucleotide or additional oligonucleotide is an immunomodulatory (i.e., immunostimulatory or immunosuppressive) oligonucleotide. In some embodiments, the immunomodulatory oligonucleotide comprises a CpG motif. Oligonucleotides that contain CpG motifs that are similar to those found in bacterial DNA stimulate a similar response of the TLR receptors. Therefore immunomodulatory oligonucleotides have various potential therapeutic uses, including treatment of immune deficiency and cancer.

Protein therapeutics include, without limitation peptides, antibodies, enzymes, structural proteins, receptors and other cellular or circulating proteins as well as fragments and derivatives thereof. Specific proteins contemplated by the disclosure include, without limitation, transforming growth factor beta 3 (TGF-β3), interferon alpha, a collagenase, and/or TNF-stimulated gene-6 (TSG-6).

In some embodiments, agents include small molecules. The term “small molecule,” as used herein, refers to a chemical compound, for instance a peptidometic that may optionally be derivatized, or any other low molecular weight organic compound, either natural or synthetic. Such small molecules may be a therapeutically deliverable substance or may be further derivatized to facilitate delivery. Small molecules contemplated by the disclosure include, without limitation, imiquimod, RepSox, bleomycin, allantoin, oleanolic acid, honokiol, a statin, and/or heparin.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, or about 1000 Daltons.

Compositions. The disclosure provides compositions that comprise a pharmaceutically acceptable carrier and a spherical nucleic acid (SNA) of the disclosure. The term “carrier” refers to a vehicle within which the SNA is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances include but are not limited to polymers (e.g., polyethylene glycol), carbohydrates (e.g., starch, glucose, lactose, sucrose, or cellulose), and alcohols (e.g., glycerol, sorbitol, or xylitol).

Adjuvants contemplated by the disclosure include but are not limited to emulsions, microparticles, immune stimulating complexes (iscoms), LPS, CpG, or MPL.

Topical administration. The disclosure provides compositions comprising a SNA that are administered topically to treat and/or attenuate an abnormal scar in a subject. For topical administration, it is contemplated that in some embodiments a composition of the disclosure comprises a vehicle.

Vehicles useful in the compositions and methods of the present disclosure are known to those of ordinary skill in the art and include without limitation an ointment, cream, lotion, gel, foam, buffer solution, or water. In some embodiments, a vehicle does not include water. In some embodiments, vehicles comprise one or more additional substances including but not limited to salicylic acid, alpha-hydroxy acids, or urea that enhance the penetration through the stratum corneum.

In various aspects, vehicles contemplated for use in the compositions and methods of the present disclosure include, but are not limited to, Aquaphor® healing ointment, A+D, polyethylene glycol (PEG), glycerol, mineral oil, Vaseline Intensive Care cream (comprising mineral oil and glycerin), petroleum jelly, DML (comprising petrolatum, glycerin and PEG 20), DML (comprising petrolatum, glycerin and PEG 100), Eucerin moisturizing cream, Cetaphil (comprising petrolatum, glycerol and PEG 30), Cetaphil, CeraVe (comprising petrolatum and glycerin), CeraVe (comprising glycerin, EDTA and cholesterol), Jergens (comprising petrolatum, glycerin and mineral oil), and Nivea (comprising petrolatum, glycerin and mineral oil). One of ordinary skill in the art will understand from the above list that additional vehicles are useful in the compositions and methods of the present disclosure.

An ointment, as used herein, is a formulation of water in oil. A cream as used herein is a formulation of oil in water. In general, a lotion has more water than a cream or an ointment; a gel comprises alcohol, and a foam is a substance that is formed by trapping gas bubbles in a liquid. These terms are understood by those of ordinary skill in the art.

Abnormal scarring. The disclosure provides compositions comprising a SNA that are administered topically to treat and/or attenuate an abnormal scar in a subject. By “abnormal scar” is meant a scar that is defined by excessive collagen deposition during wound healing, leading to an area of skin which is firmer, and more elevated than the surrounding skin.

Abnormal scars include, without limitation, hypertrophic scars and keloid scars.

### EXAMPLES

### Example 1

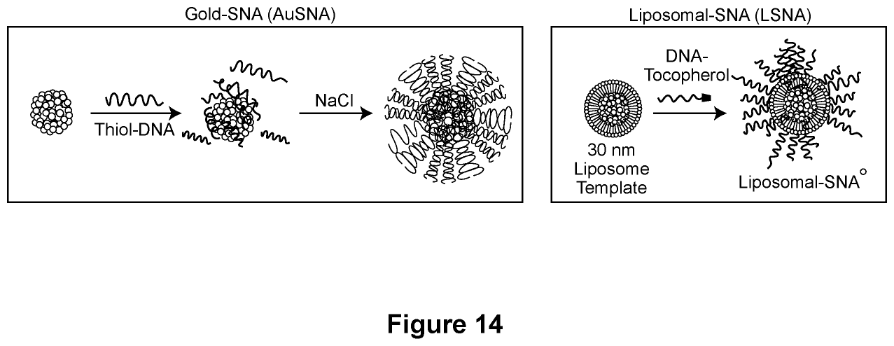

Specifically, three SNA constructs, including gold SNAs (AuSNAs), liposomal SNAs (LSNAs), and micellular SNAs (MSNAs), targeting transforming growth factor 1 (TGF-β1) were prepared and characterized. Hydrodynamic diameter and zeta potential of nanoparticles and SNAs were measured by a Zetasizer utilizing dynamic light scattering with a 660 nm laser source. As-synthesized particles were diluted 1:100 with nanopore water before measurement. Graphical depictions of exemplary AuSNAs and LSNAs are shown in FIG. 14.

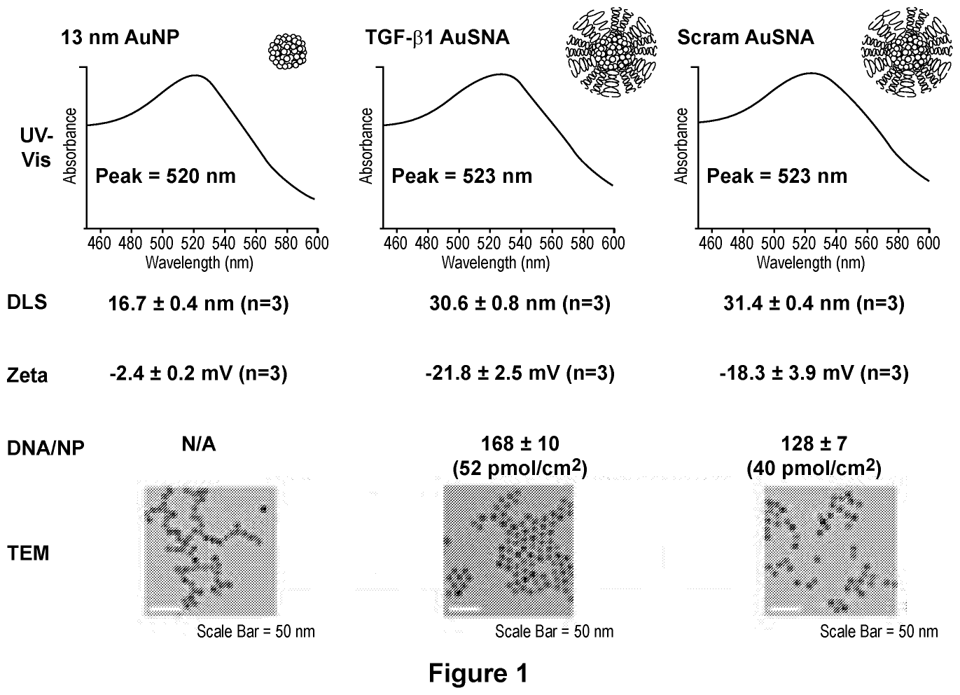

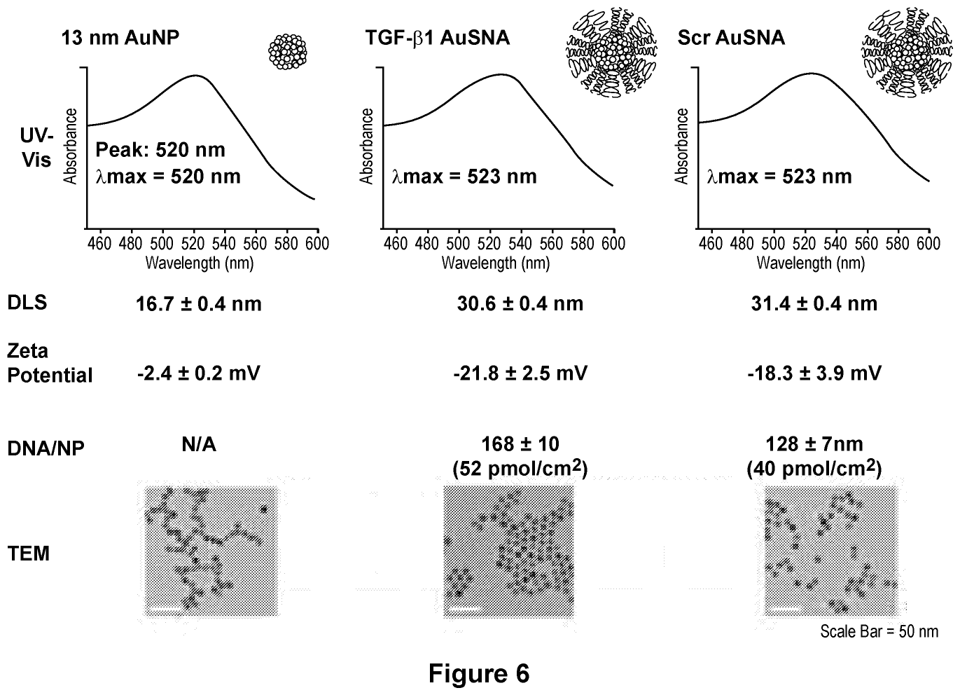

To measure the number of strands chemically attached to Au nanoparticles, AuSNAs were first diluted to 1 nM by Au and then dissolved with equal volume of 40 mM KCN. The mixture was incubated until AuSNAs were fully dissolved. DNA quantification of the resulting solution was done using the Quant-iT™ OliGreen™ ssDNA Assay Kit and further verified by a UV-Vis spectrophotometer, and then the concentration of oligonucleotides was determined by Beers' law with extinction coefficients of each oligonucleotide. See FIGS. 1 and 6.

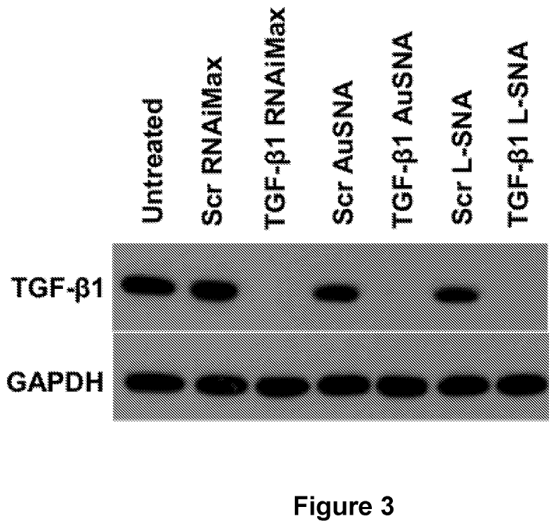

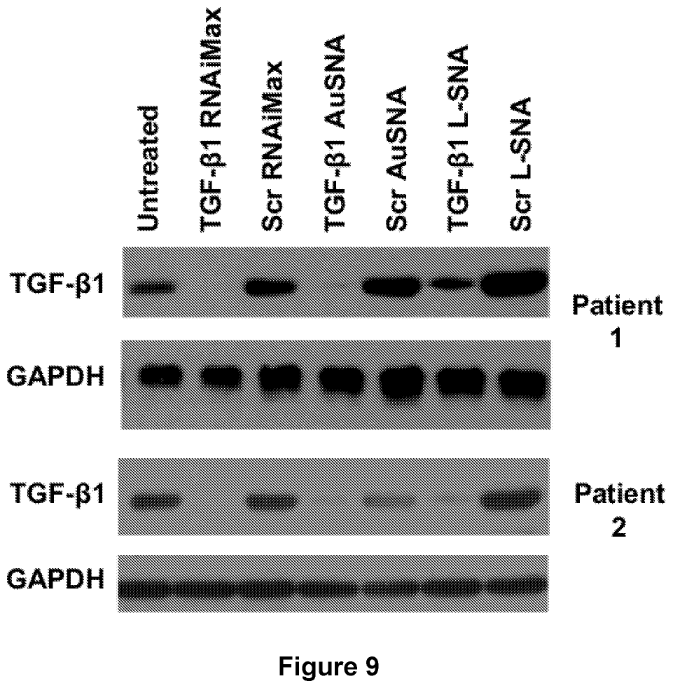

In vitro and in vivo studies showed that these constructs significantly suppress the expression level of TGF-β1, a protein central to abnormal scar formation, at both the mRNA and protein level. HSF cells were seeded into a well plate and allowed to adhere for 12 hours. They were then treated with 1 μM of SNAs by DNA in Opti-MEM for 12 hours. After 12 hours, an equal volume of MEM media with 20% fetal bovine serum (FBS) and 1% penstrap was added on top of the SNA-containing media. After 36 hours, that media was changed to MEM media with 10% FBS and 1% penstrap. After 12 hours the cells were lysed with a RIPA buffer cocktail containing 1:100 protease inhibitor cocktail, 1 mM PMSF (phenylmethylsulfonyl fluoride), 1 mM NaF, and 2 mM sodium orthovanadate. Protein amount was equalized and run in a gradient cell. The protein was then transferred to a nitrocellulose membrane and probed with anti-TGF-β1 antibody, and anti-GAPDH antibody as a loading control. Bands were imaged with chemiluminescence if HRP-tagged secondary antibodies and exposed onto X-ray film. Downstream signaling of TGF-β1 was totally abolished due to knockdown of TGF-β1. See FIG. 3. The experiment was then repeated with KF cells instead of HSF cells, and cells were gathered from two different patients. Results are shown in FIG. 9.

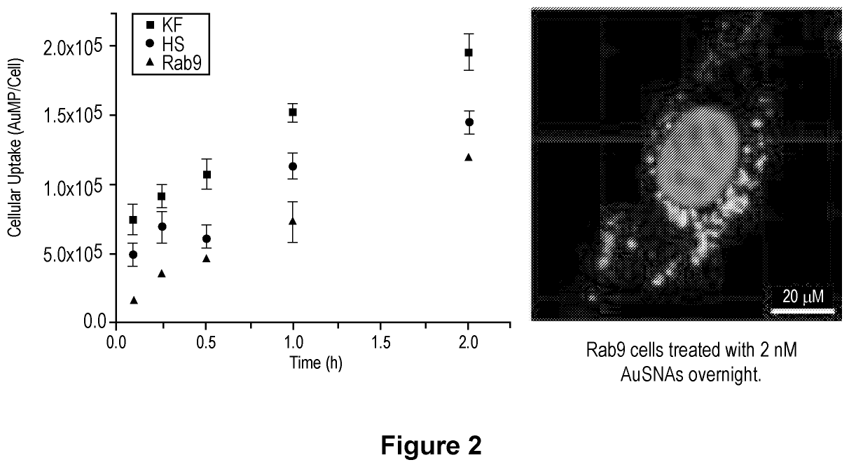

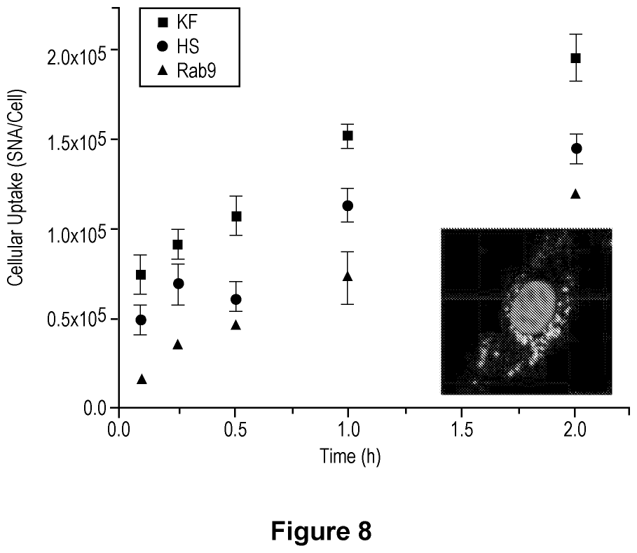

Three cell lines were seeded into a 12-well plate: rabbit fibroblasts (Rab9), human hypertrophic scar-derived fibroblasts (HSF), and human keloid scar-derived fibroblasts (KF), and allowed to adhere for 12 hours. Afterwards, each cell line was incubated with 10 nM AuSNAs in Opti-MEM media. At the various time points shown on the graph, the cells were washed, dissociated from the well plate, and digested in a 97%/3% v %/v % nitric acid/hydrochloric acid solution overnight. These solutions were then diluted and analyzed for gold content using inductively coupled plasma-mass spectrometry (ICP-MS). See FIGS. 2 and 8.

For confocal microscopy, Rab9 cells were seeded into a confocal dish and allowed to adhere. They were then treated for fluorescently-tagged DNA SNAs for 12 hours in Opti-MEM media at a 100 nM by fluorescently-tagged DNA. The cells were then washed and subsequently fixed using a 3.7% formaldehyde solution in PBS for 10 minutes. The cells were then stained with a DAPI nuclear stain and finally imaged with confocal microscopy. See FIGS. 2 and 8.

### Example 2

### Screen Antisense DNA that Knocks Down TGF-β1

To evaluate TGF-β1 mRNA expression level, Rab9 fibroblasts were seeded into 96-well plates and allowed to adhere overnight. Antisense sequences against TGF-β1 were added to the cells at a concentration of 1 μM in Opti-MEM and with a transfection reagent. After a 12-hour incubation, the media was changed to MEM with 10% fetal bovine serum and 1% penstrap. To quantify gene expression, total RNA was extracted from cells plated in 96-well plates using the RNeasy 96 well plate kit per the manufacturer's protocol. RNA was subsequently reverse transcribed to generate cDNA using the High-Capacity cDNA reverse Transcription Kit. cDNA was mixed with Roche's Lightcycler 480 Probe Master Mix along with probes and primers (per manufacturer's protocol). GAPDH was used as a housekeeping gene with the primers and probes generated in house using the following sequences: Forward—5′-CAA GGT CAT CCA TGA CAA CTT TG-3′ (SEQ ID NO: 1), Reverse—5′-GGG CCA TCC ACA GTC TTC T-3′ (SEQ ID NO: 2), Probe—5′-HEX-ACC ACA GTC CAT GCC ATC ACT GCC A—BHQ1 (SEQ ID NO: 3). All other primers/probes were obtained from Life Technologies. qRT-PCR was performed on a Roche Lightcycler 480 and the relative abundance of each mRNA transcript was normalized to GAPDH expression.

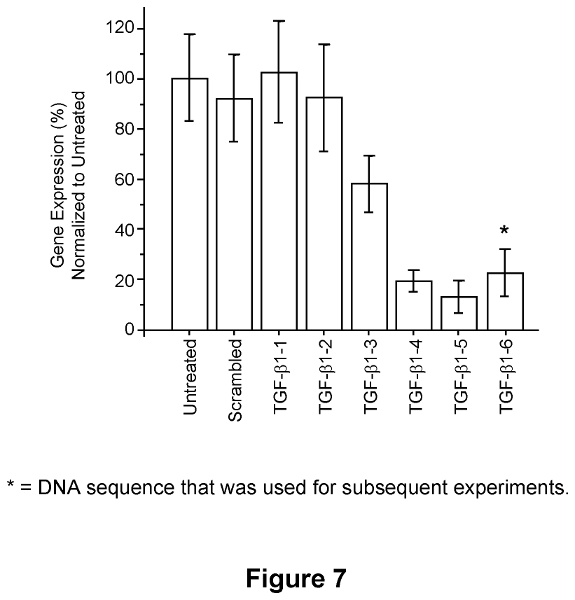

FIG. 7 depicts the results of the experiments, which indicate that the sequences were efficient at inhibiting expression of TGF-β1.

### Example 3

### Evaluation of SNA Efficacy to Reduce Abnormal Scarring in a Rabbit Ear Model

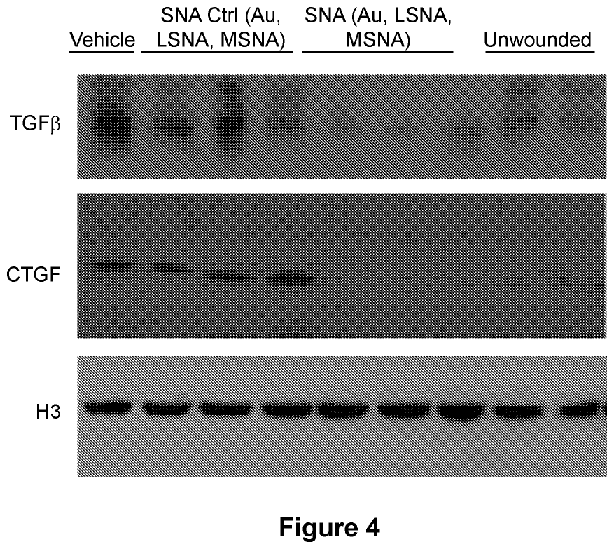

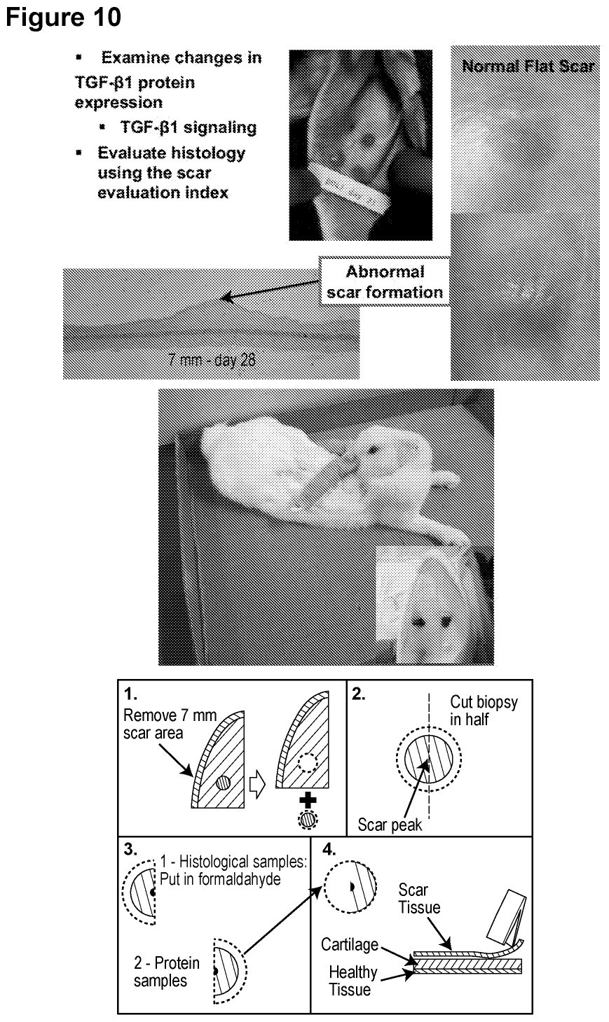

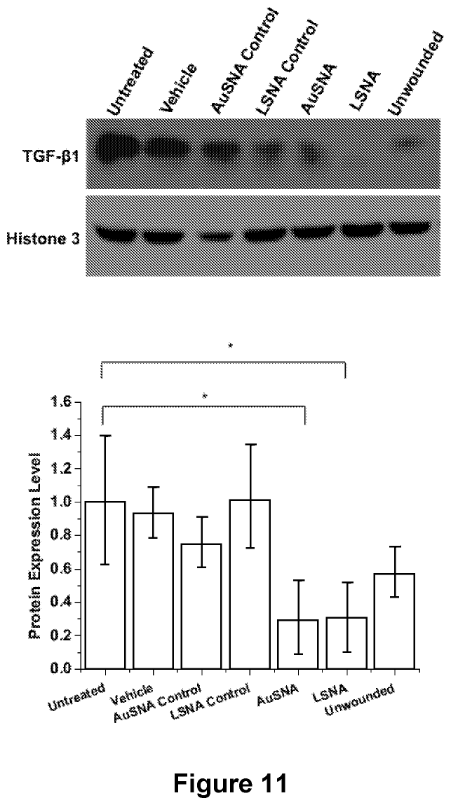

Rabbit Studies: New Zealand white rabbits were used for this study. Four, 7 mm punch wounds were made on the front of each rabbit ear. The wounds extended down to the cartilage of the ear. The wounds were allowed to heal for approximately two weeks, or until all of the wounds were closed. After the wounds were closed, the resulting scars were topically treated with 20 mg of a 500 nM SNA-in-Aquaphor mixture (50/50 wt/wt). There were 8 experimental conditions in total, and each rabbit had a scar which was treated with one of those conditions. This treatment was repeated three times a week for six weeks. After completion of treatment, the rabbits were sacrificed and the treated scars were punched out of each ear. An additional punch was taken from an unscarred region of each ear to represent the untreated group. The punch biopsies were then cut into near semi-circles, with one half a bit larger than the other in order to include the entire scar center. The half with the scar center was formalin fixed and paraffin embedded (FFPE) in order to be used for subsequent histological analysis. The other portion was lysed in order to perform subsequent Western blot analysis. See FIG. 10 for depiction of experimental protocol, and FIGS. 4, 5, and 11 for results.

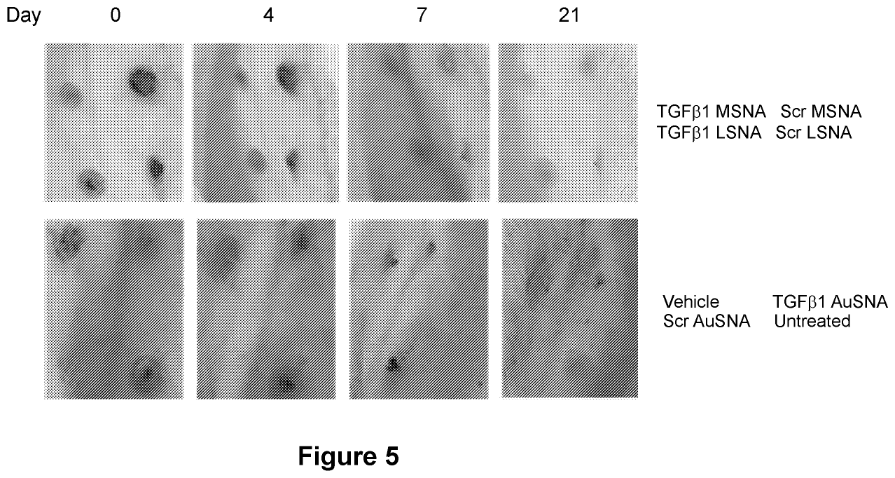

In some embodiments, compositions of the disclosure treat or attenuate abnormal scars. In further embodiments, the scar is a hypertrophic scar or a keloid scar. As shown herein, SNA-treated scars showed that SNA treatment improves histology of the scar, compared to control treatment. See FIG. 5.

### Example 4

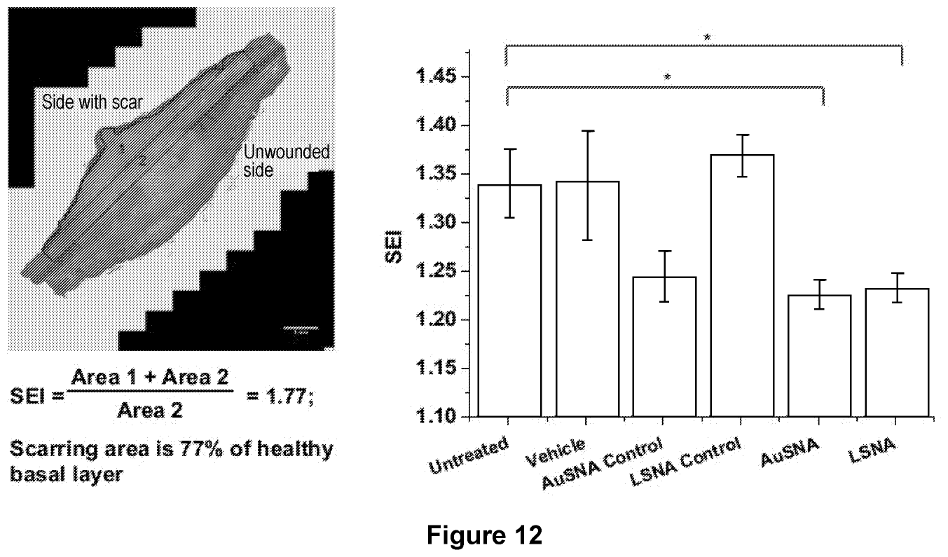

Harvested scar tissues were sectioned into 5 μm slice at the Northwestern Mouse Histology and Phenotyping Laboratory, followed by H&E staining. H&E stained tissue samples were embedded onto a glass slide. Light microscopy images were taken using a fluorescent microscope (Leica DM6B Widefield) with 10× magnification. Determination of scar area was performed under the supervision of two dermatology doctors from Northwestern using Image J. See FIG. 12, which shows that treatment with both SNA constructs resulted in reduced scar elevation.

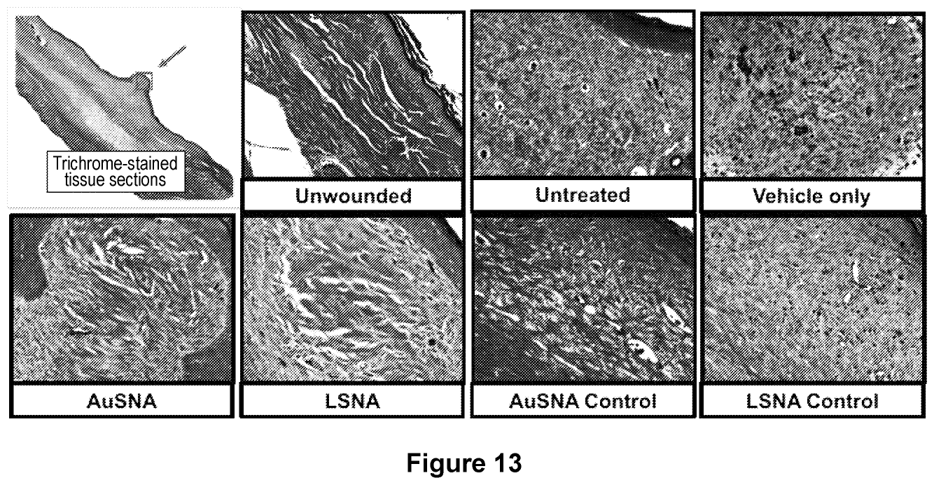

Harvested scar tissues were sectioned into 5 μm slice at the Northwestern Mouse Histology and Phenotyping Laboratory, followed by trichrome staining. Trichrome-stained tissue samples were embedded onto a glass slide. Light microscopy image was taken using a fluorescent microscope (Leica DM6B Widefield) with 10× magnification. Results are shown in FIG. 13 and demonstrate that SNA treatment leads to collagen reformation.

The figures illustrate various embodiments contemplated by the present disclosure. The figures are exemplary in nature and are in no way intended to be limiting.

### CONCLUSIONS

SNA constructs showed potent downregulation of TGF-β1 in vitro.
In vitro knockdown was translated to in vivo TGF-β1 protein suppression in a rabbit ear model.
Histological analysis shows that AuSNA and LSNA constructs significantly reduce scar elevation (P<0.05).

What is claimed is:

1. A method of treating and/or attenuating an abnormal scar in a subject, comprising topically administering a composition to the abnormal scar, the composition comprising:
a spherical nucleic acid (SNA) comprising a nanoparticle and an oligonucleotide on the surface of the nanoparticle,
wherein topical administration of the SNA inhibits expression of transforming growth factor beta 1 (TGF-β1), thereby treating and/or attenuating the abnormal scar.

2. The method of claim 1, wherein the nanoparticle is organic.

3. The method of claim 1, wherein the nanoparticle is inorganic.

4. The method of claim 1 or claim 2, wherein the nanoparticle is a liposome.

5. The method of claim 4, wherein the liposome comprises a lipid selected from the group consisting of 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, and lipid A.

6. The method of any one of claim 1-2 or 4-5, wherein the oligonucleotide comprises a tocopherol, a cholesterol moiety, DOPE-butamide-phenylmaleimido, or lyso-phosphoethanolamine-butamide-pneylmaleimido.

7. The method of claim 1 or claim 2, wherein the nanoparticle is a micelle.

8. The method of claim 1 or claim 2, wherein the nanoparticle is polymeric.

9. The method of claim 8, wherein the nanoparticle comprises poly (lactic-co-glycolic acid)(PLGA).

10. The method of claim 1 or claim 3, wherein the nanoparticle is metallic.

11. The method of claim 10, wherein the nanoparticle is a colloidal metal.

12. The method of claim 11, wherein the nanoparticle is selected from the group consisting of a gold nanoparticle, a silver nanoparticle, a platinum nanoparticle, an aluminum nanoparticle, a palladium nanoparticle, a copper nanoparticle, a cobalt nanoparticle, an indium nanoparticle, and a nickel nanoparticle.

13. The method of any one of claims 10-12, wherein the oligonucleotide is bound to said nanoparticle through one or more sulfur linkages.

14. The method of any one of claims 1-13, wherein the oligonucleotide is from about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, or about 5 to about 10 nucleotides in length.

15. The method of any one of claims 1-14, wherein the oligonucleotide comprises RNA or DNA.

16. The method of claim 15, wherein the RNA is selected from the group consisting of a small inhibitory RNA (siRNA), a single-stranded RNA (ssRNA) that forms a triplex with double stranded DNA, and a ribozyme.

17. The method of claim 15, wherein the RNA is a microRNA.

18. The method of claim 15, wherein the DNA is antisense-DNA or DNAzyme.

19. The method of any one of claims 1-18, wherein the nanoparticle ranges from about 1 nm to about 250 nm in diameter, about 1 nm to about 240 nm in diameter, about 1 nm to about 230 nm in diameter, about 1 nm to about 220 nm in diameter, about 1 nm to about 210 nm in diameter, about 1 nm to about 200 nm in diameter, about 1 nm to about 190 nm in diameter, about 1 nm to about 180 nm in diameter, about 1 nm to about 170 nm in diameter, about 1 nm to about 160 nm in diameter, about 1 nm to about 150 nm in diameter, about 1 nm to about 140 nm in diameter, about 1 nm to about 130 nm in diameter, about 1 nm to about 120 nm in diameter, about 1 nm to about 110 nm in diameter, about 1 nm to about 100 nm in diameter, about 1 nm to about 90 nm in diameter, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, or about 1 nm to about 20 nm in diameter, or about 1 nm to about 10 nm in diameter.

20. The method of any one of claims 1-18, wherein the nanoparticle has a diameter of 50 nanometers or less.

21. The method of any one of claims 1-20, wherein expression of TGF-β1 is inhibited by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

22. The method of any one of claims 1-21, wherein the oligonucleotide is bound to the nanoparticle at a surface density of at least 10 pmol/cm², at least 15 pmol/cm², at least 20 pmol/cm², at least 10 pmol/cm², at least 25 pmol/cm², at least 30 pmol/cm², at least 35 pmol/cm², at least 40 pmol/cm², at least 45 pmol/cm², or at least 50 pmol/cm².

23. The method of any one of claims 1-21, wherein the nanoparticle comprises from about 50 to about 500 oligonucleotides.

24. The method of claim 23, wherein the particle comprises 150 to 350 oligonucleotides.

25. The method of claim 23, wherein the particle comprises 200 to 300 oligonucleotides.

26. The method of any one of claims 1-25, wherein the SNA further comprises a therapeutic.

27. The method of claim 26, wherein the therapeutic is encapsulated in the nanoparticle.

28. The method of claim 26, wherein the therapeutic is conjugated to the surface of the nanoparticle.

29. The method of any one of claims 26-28, wherein the therapeutic is a small molecule, an additional oligonucleotide, a protein, or a peptide.

30. The method of claim 29, wherein the protein is a steroid or an antibody.

31. The method of claim 29, wherein the antibody is directed against transforming growth factor beta receptor 1 (TGFBR1).

32. The method of claim 29, wherein the additional oligonucleotide is siRNA, a ribozyme, antisense DNA, or DNAzyme.
